(19) United States
(12) Patent Application Publication
(10) Pub. No.: US 2002/0065500 A1
(43) Pub. Date: May 30, 2002

(54) Diaper enclosure housing disposable plastic bag

(76) Inventor: Paul J. Rossi, Waltham, MA (US)

Correspondence Address: Paul J. Rossi, 110 Worcester Lane, Waltham, MA, 02451, US

(21) Appl. No.: 09/803,826

(22) Filed: Mar. 12, 2001

Related U.S. Application Data
(63) Continuation-in-part of application No. 09/632,556, filed on Nov. 13, 2000.

(51) Int. Cl. A61F 13/15

(52) U.S. Cl. 604/385.06

(57) ABSTRACT

Currently when a diaper/pull-up is soiled and removed, it is typically folded into itself, covering the waste, and thrown directly into the trash or placed into a plastic bag and then thrown into the trash. However, there are a numerous times when a trash container or plastic bag is not easily accessible. This invention would allow for a tear open enclosure housing a disposable plastic bag to be located directly on the diaper/pull-up allowing the diaper to be stored in this disposable plastic bag and discarded at a later time.

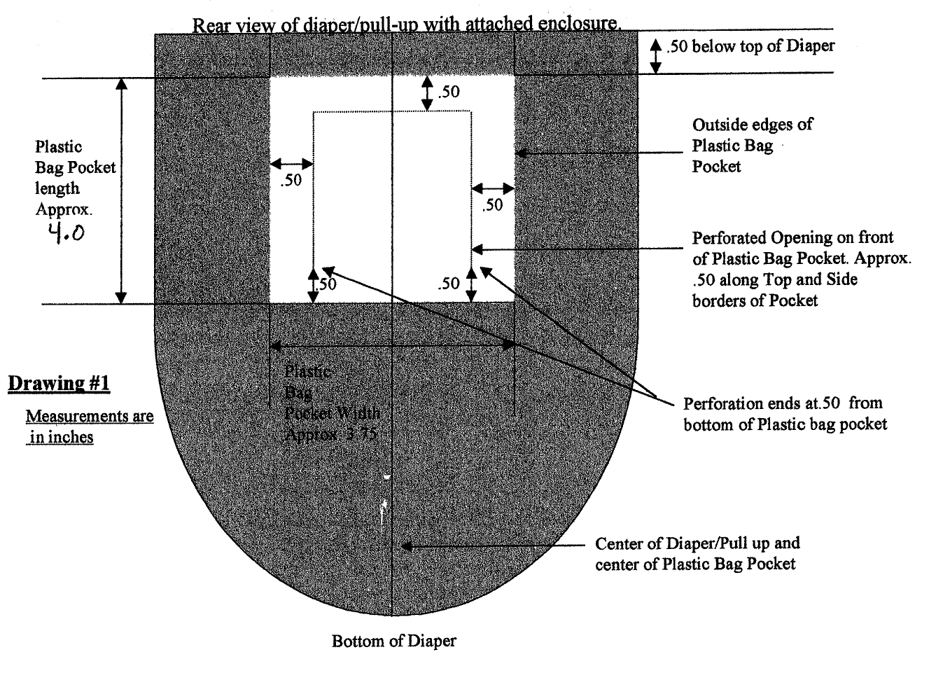

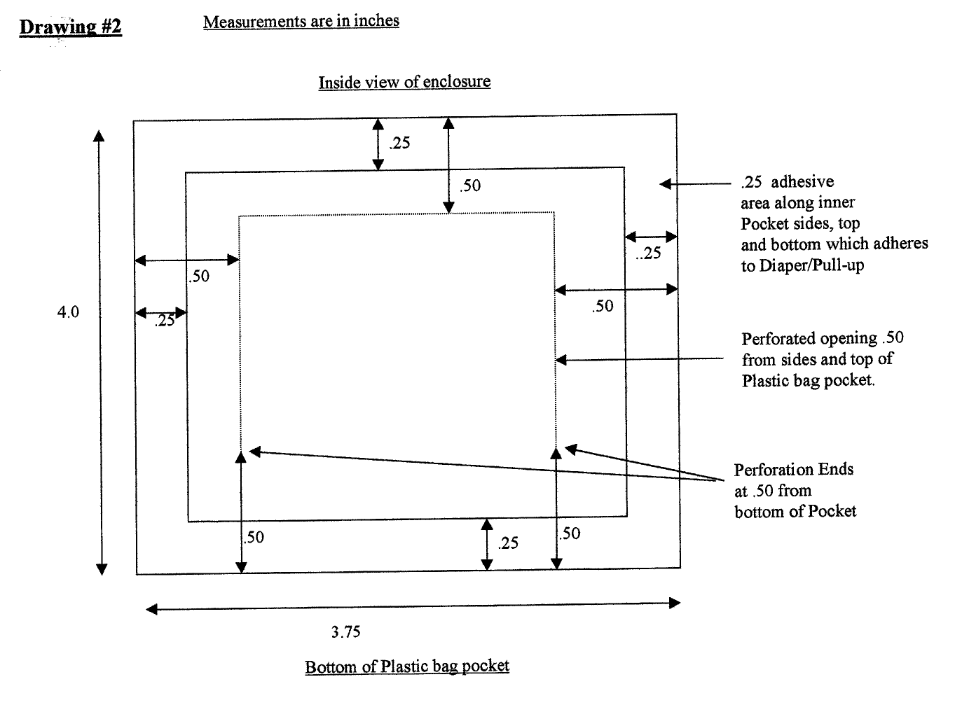

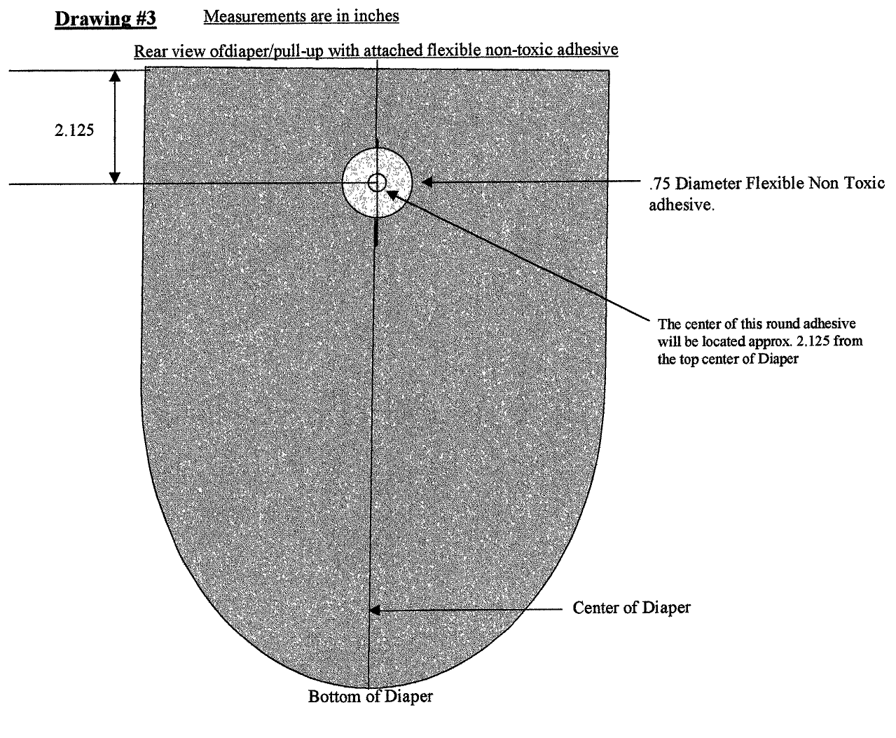

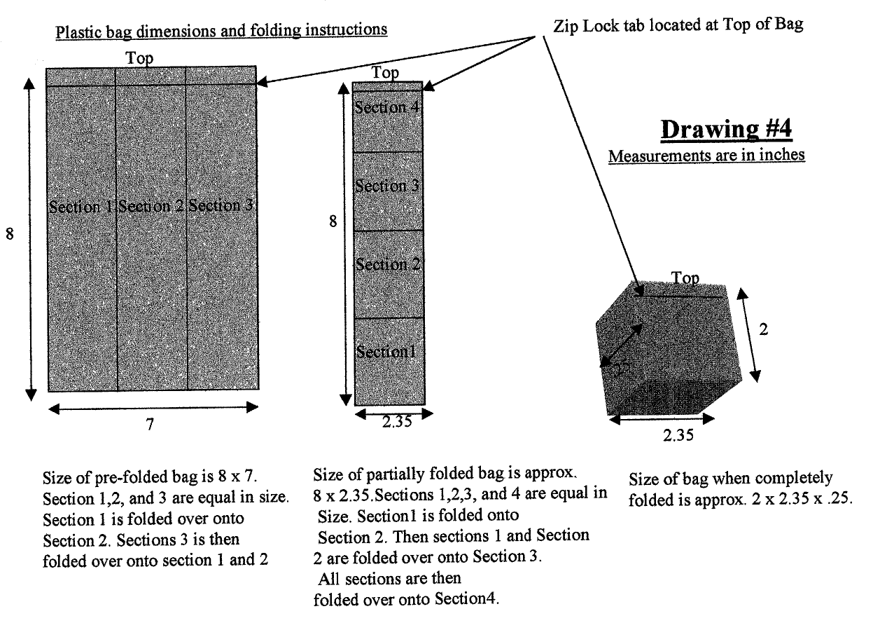

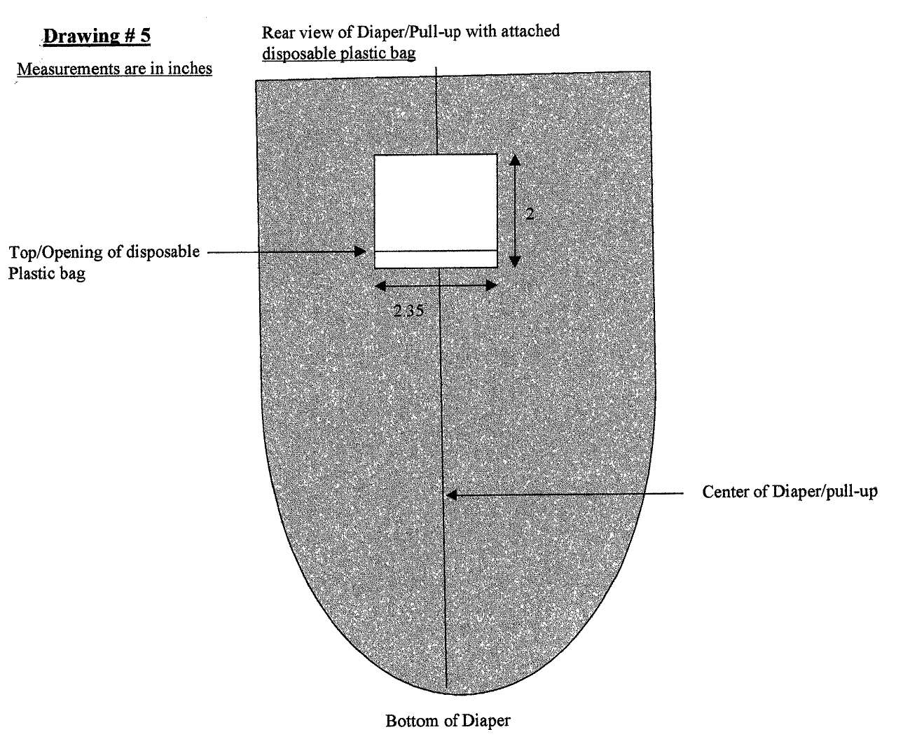

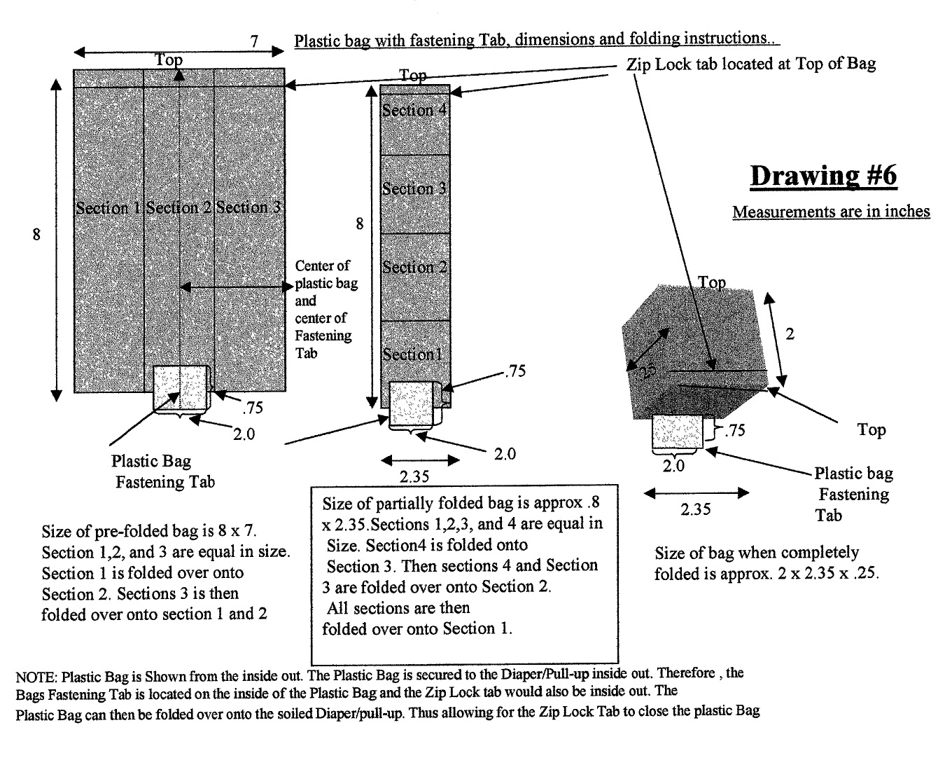

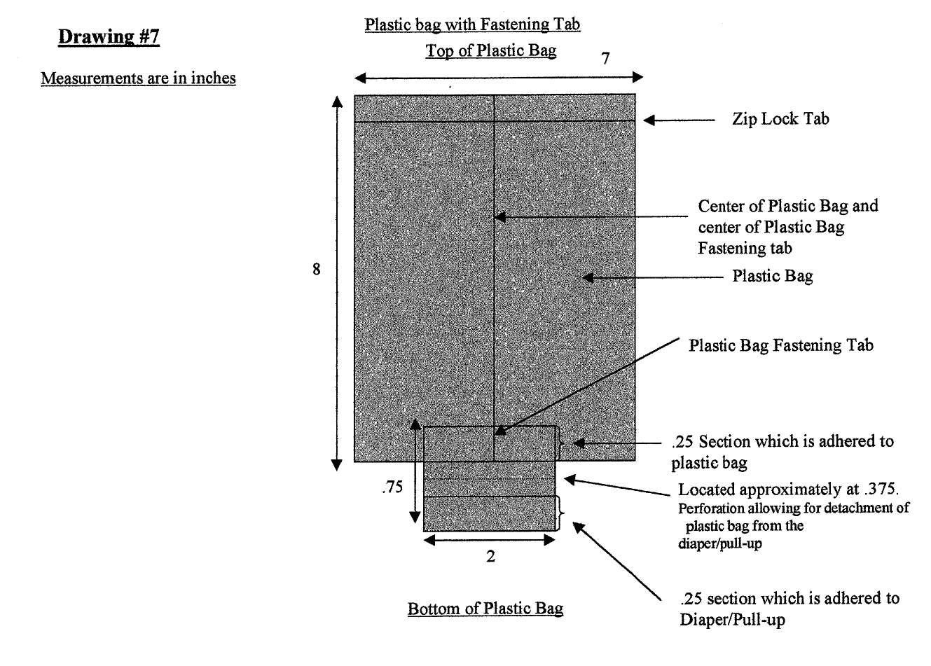

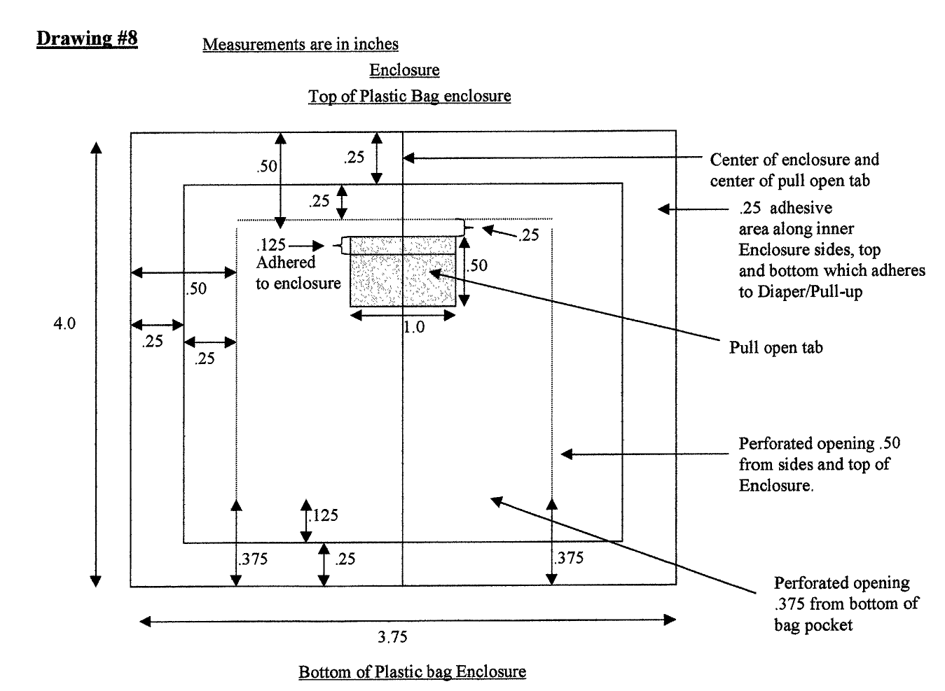

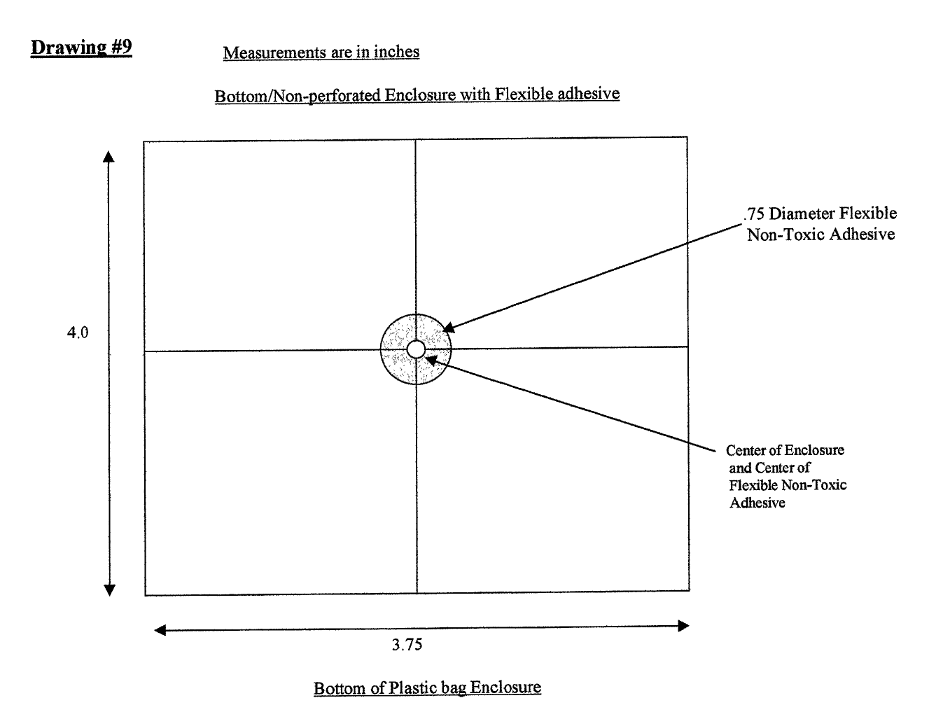

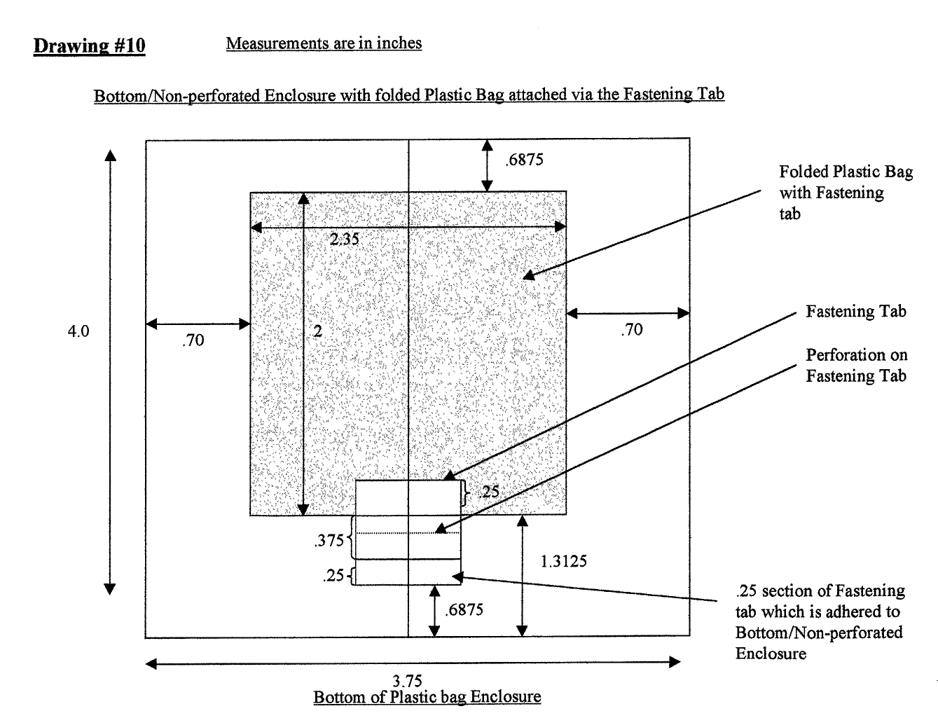

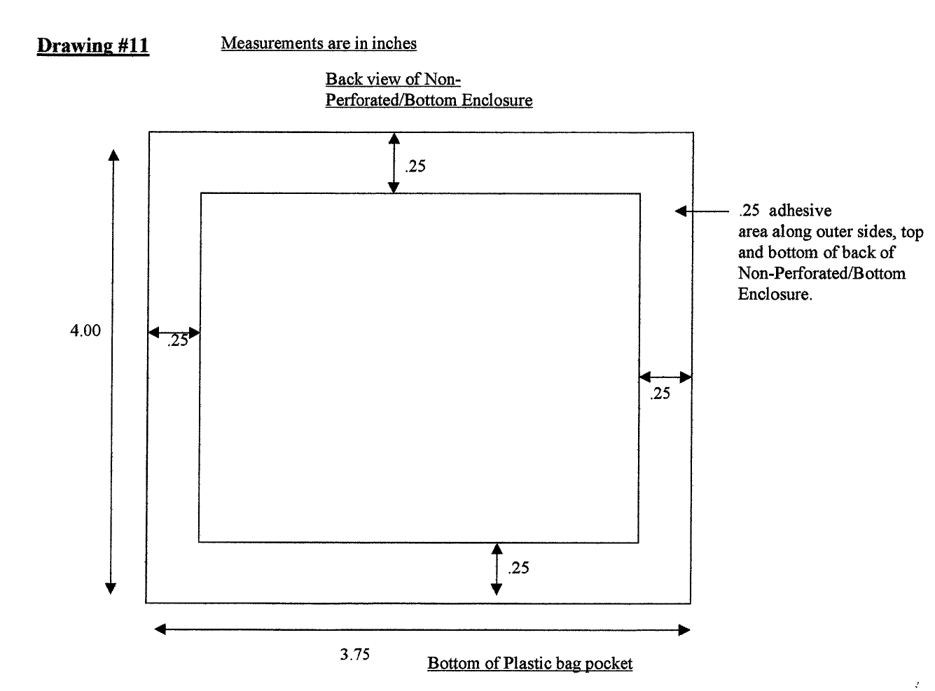

This invention is a continuation-in-part to application Ser. No. 09/632,556

### DESCRIPTION OF INVENTION, IN DETAIL WHICH I AM SEEKING TO PATENT

The present invention relates to a diaper/pull-up with a built-in disposable plastic bag. This invention would apply to all types of diapers and pull-ups, for all ages: new-born to toddler. The invention consists of a thin clear plastic bag located in the rear center of the diaper/pull-up, which is housed in a tear open enclosure.

Currently when a diaper/pull-up is soiled and removed, it is typically folded into itself, covering the waste, and thrown directly into the trash or placed into a plastic bag and then thrown into the trash. However, there are a numerous times when a trash container or plastic bag is not easily accessible. This invention would allow for a disposable plastic bag to be located directly on the diaper and easily removed allowing the diaper to be stored in the bag and discarded at a later time.

The plastic bag may include a resealable closure such as a tongue-in-grove structure known to the art as Zip-lock RTm, or an adhesive. The bag could also have an embossed top for better handling. The bag dimensions would be as follows, 8″height×7″length and would be single layer and fairly thin. The bag would be folded in such a way as to maintain a final approximate dimension of 2″height×2.35″width×0.25″depth (See Drawing No. 4 for folding instructions and dimensions). The plastic bag would be held in place on the diaper/pull-up by 0.75″ diameter flexible non-toxic adhesive located at the rear center of the diaper/pull-up approximately 2.125″ from the top of the diaper/pull-up (See drawing No. 3). The adhesive would adhere to the diaper surface and will allow for the plastic bag to be removed from the diaper, leaving the adhesive on the diaper/pull-up and not on the disposable plastic bag. The adhesive would be placed on the diaper during the manufacturing process. The center of the folded plastic bag would be immediately placed onto the adhesive with the resealable opening of the plastic bag lying horizontally along the diaper and facing toward the bottom of the diaper (See drawing No. 5).

A second method of attaching the Plastic Bag to the Diaper/Pull-up would involve a permanent/non removable Fastening Tab located on the bottom inside of the disposable Plastic Bag. The Fastening Tab would have one portion attached to the plastic bag and one portion attached to the Diaper/Pull-up (See drawings #6 and #7). This Fastening Tab could also have a perforation in its center to allow for the plastic bag to be separated from the diaper (See drawing #7). The plastic bag would be folded as described in drawing #6 and then be attached to the diaper/pull-up inside out The Zip lock Tab and the Fastening Tab, which are located in the interior of the plastic bag, would be facing out towards the insides of the Enclosure Perforated Top and the Enclosure Non-Perforated Bottom. The soiled Diaper would then be folded into the plastic bag. Thus allowing the Fastening Tab and the Zip Lock Tab to be on the inside of the bag and for the Zip Lock tab to close properly (See Drawings #6 and #7).

After the plastic bag is attached to the Diaper/pull-up using either of the above attachment methods, an Enclosure compartment made of polyurethane would be placed over the plastic bag, enclosing the entire bag. The enclosure would have the following dimensions 3.75height×3.75length×0.005 thickness, single layer. The enclosure will have a 0.25″area along the top, bottom, and sides along the inside. This 0.25″area will have a permanently sealing adhesive applied to it. The center of the enclosure will then be placed directly over the center of the plastic bag and secured to the diaper via the adhesive located on the 0.25″area along the borders of the inside of the enclosure. The enclosure will be perforated along the top, and sides, approximately 0.50″from the edge of the enclosure (See Drawing No. 2). The bottom of the enclosure would not be perforated thus allowing the user to tear open the compartment and remove the plastic bag, leaving the opening to the enclosure attached to the diaper/pull-up. The enclosure could allow for a perforation along the bottom (See Drawing #8), in addition to the top and sides, allowing for the center of the enclosure to be discarded into the plastic bag. The tear open compartment could also have an Opening Tab attached to it so as to allow a consumer to grasp the Opening Tab and open the compartment. The Opening Tab would be secured, via non-toxic permanent adhesive, at approximately 0.25 inches from the top of the perforated compartment (See Drawing #8). The adhesive would be placed along the top of he tab, approximately 0.25 inches in width and running along the entire 1 inch of the tab (See Drawing #8).

A second Enclosure method could also be employed. The Top Enclosure section with the Perforated opening Tab would be attached to a Bottom Enclosure section which does not have a Perforated opening. This Bottom/Non-perforated Enclosure section could be made of the same material and would have the same dimensions as the Top Enclosure/perforated section (See Drawing #9). The Disposable Plastic Bag would be folded as identified/described in Drawing #4. The Disposable Plastic Bag would then be attached to the inside of the Bottom/Non-Perforated Enclosure section using either of the methods described earlier; 0.75 inch Flexible non-toxic adhesive (See Drawing #9) or Fastening Tab (See Drawing #10). This final Enclosure, consisting of the Top Perforated Enclosure, the Disposable Plastic bag, and the Bottom non-perforated Enclosure would then be adhered to the Diaper/Pull-up via a 0.25 Permanent non-toxic adhesive located on the back of the Bottom/Non-perforated Diaper/Pull-up at 0.50 inches from the top center as described/identified in Drawing #1.

The plastic bag dimensions, color, material and closing apparatus may vary depending upon the size of the diaper/pull-ups, manufacturing, marketing, and safety requirements.

The flexible non-toxic adhesives and permanent non-toxic adhesives may vary based on manufacturing, marketing, and safety requirements.

The enclosure material, dimensions, and perforation locations may vary based on manufacturing, marketing, and safety requirements.

The plastic Bag Fastening Tab and the perforated compartment Opening Tab dimensions, color, material, and location may vary depending on the size of the diaper/pull-up, manufacturing, marketing and safety requirements.

Thus what has been described above is a diaper/pull-up with a built in disposable plastic bag which is housed in a tear open enclosure.

While the present invention has been described with what are presently considered to be the most practical and preferred embodiments and methods, the invention is not limited to the disclosed embodiments or processes.

Drawing Listing and Description: (Note all Drawing Dimensions are Depicted in Inches)

Drawing No. 1: Rear view of diaper/pull-up with enclosure.

Drawing No. 2: Inside view of enclosure.

Drawing No. 3: Rear view of diaper/pull-up with attached flexible non-toxic adhesive.

Drawing No. 4: Plastic bag dimensions and folding instructions.

Drawing No. 5: Rear view of diaper/pull-up with attached disposable plastic bag.

Drawing No. 6: Plastic Bag with fastening Tab, dimensions and folding instructions.

Drawing No. 7: Plastic bag with Fastening Tab

Drawing No. 8: Enclosure with Opening Tab

Drawing No. 9: Bottom/Non-Perforated Enclosure with Flexible Adhesive

Drawing No. 10: Bottom/Non-Perforated Enclosure with Fastening Tab

Drawing No. 11: Back view of Bottom/Non-Perforated Enclosure Section

1. What I claim as my invention is a tear open Enclosure housing a built-in disposable plastic bag located in the rear center of a Diaper or pull-up. The Disposable Plastic Bag can be attached directly to the Diaper/Pull-up via a Flexible non-toxic adhesive or by a Fastening Tab and then enclosed by an accessible Perforated Enclosure. The Disposable Plastic Bag could also be attached to a Non-Perforated enclosure, which would then be covered by an accessible Perforated Enclosure, this entire enclosure can then be attached to the diaper/pull-up.
